(19) United States
(12) Patent Application Publication
(10) Pub. No.: US 2024/0315675 A1
(43) Pub. Date: Sep. 26, 2024

(54) BIOPSY DEVICE AND METHOD FOR SAMPLING CELLS OR TISSUE IN MAMMALS

(71) Applicant: Extendo Medical AB, Arsta (SE)

(72) Inventors: Mikael SANDELL, Årsta (SE); Niclas ROXHED, Bromma (SE); Göran STEMME, Lidingö (SE); Staffan HOLMIN, Bromma (SE); Stefan JONSSON, Sollentuna (SE); Wouter METSOLA VAN DER WIJNGAART, Sollentuna (SE)

(21) Appl. No.: 18/271,823

(22) PCT Filed: Jan. 18, 2022
(86) PCT No.: PCT/SE2022/050052

§ 371 Date: Jul. 11, 2023

(30) Foreign Application Priority Data
Jan. 18, 2021 (SE) 2130017-3

(51) Int. Cl. A61B 10/02

(52) U.S. Cl. CPC A61B 10/0233; A61B 2010/0216

(57) ABSTRACT

A biopsy device for sampling cells or tissue in a subject, the biopsy device comprising: a tubular member defining a lumen; and an elongated member movably arranged within the lumen of the tubular member, wherein a distal portion of the elongated member comprises a tissue capture arrangement including a plurality of rigid protrusions.

### TECHNICAL FIELD

The present disclosure generally relates to a biopsy device and method for sampling cells or tissue in a subject. More particularly, the biopsy device and method may be used for sampling endothelial cells in a blood vessel or lymphatic vessel.

### BACKGROUND ART

Endothelial cells (ECs) line the inner wall of blood vessels and lymphatic vessels and are important in maintaining correct circulatory function and in transferring molecules from the blood or lymph to the surrounding tissue. Incorrect function of this cell layer can lead to a variety of diseases, such as atherosclerosis and hypertension. The ECs have phenotypic variations in different parts of the body, and sometimes even in different parts of the same vasculature. If these cells could be selectively harvested, a lot of valuable information could be gathered on vascular disease conditions. Previous publications focus on sampling of endothelial cells using different tools, circulating ECs or disease conditions. The devices used are mostly guidewires, stents or coils navigated inside vessels. These devices have no inherent capturing mechanism, leading to high variation in the sampled amount.

US 2017/0181733 A1 discloses a system and a method for sampling endothelial cells within pulmonary vasculature by means of a cell sampling device having an elongated sheath defining an access lumen to receive an endovascular brush. The brush includes brush elements coupled to a shaft, wherein the shaft may be made of e.g. Nitinol. An actuation control is coupled to the shaft of the endovascular brush to axially translate the endovascular brush between a retracted position and an expanded position. Upon translation of the endovascular brush from the retracted position to the expanded position, the brush elements move from within the access lumen to the pulmonary vasculature to obtain endothelial cells from a target artery of the pulmonary vasculature.

WO 2006/116019 A2 discloses an endovascular brush including a brush segment with brush elements in the form of a fiber, bristle, loop, ridge or corrugation configured to provide a space or spaced for retaining cells biopsied with the endovascular brush and to reduce the thrombogenicity of the endovascular brush.

However, the previously mentioned devices risks injuring the pulmonary vasculature during expansion and the brush elements may not obtain sufficient amounts of endothelial cells.

Thus, there is a need to improve the known devices and methods to overcome the disadvantages mentioned above.

### SUMMARY

An objective of the present disclosure is therefore to achieve a minimally invasive biopsy device and method which reduces tissue damage at the site of sampling whilst increasing the number of sampled cells or tissue, e.g. endothelial cells from a blood vessel or lymphatic vessel in a subject.

This objective is achieved in a first aspect of the present disclosure in which there is provided a biopsy device for sampling cells or tissue, the biopsy device comprising: a tubular member defining a lumen; and an elongated member movably arranged within the lumen of the tubular member, wherein a distal portion of the elongated member comprises a tissue capture arrangement including a plurality of rigid protrusions.

By means of the plurality of protrusions, a minimally invasive biopsy device is achieved with a high precision and increased sample yield. In this context, the term ‘rigid’ is to be understood as the protrusions being sufficiently stiff and substantially incompressible to resist movement in any direction, both in relation to each other and the surface on which they are fixed or formed. As such, the protrusions are non-expandable and will remain in the same configuration outside as well as inside the lumen of the tubular member.

In one embodiment, a height of the protrusions in a radial direction is substantially smaller than a diameter of the elongated member.

In one embodiment, a ratio between the radial height of the protrusions and the diameter of the elongated member is between 1:3 and 1:20.

In one embodiment, the radial height of the protrusions is in the range 3-100 μm.

In one embodiment, a ratio between a distance between adjacent protrusions and a radial height of the protrusions is in the range 1:2 to 5:1.

In one embodiment, a ratio between a width and a radial height of the protrusions is in the range 1:2 to 2:1.

The radial height, width and distance between the protrusions is adapted to correspond to the dimensions of the target cells or tissue to be sampled in order to reduce tissue damage whilst maximizing the amount of cells or tissue captured.

In one embodiment, the plurality of protrusions comprises a structure of pillars distributed around the circumference of the elongated member and/or along the length of the distal portion. The distribution of the protrusion over the surface of the distal portion of the elongated member greatly increases the surface area which in turn increases interaction between the biopsy device and the target sampling area, thereby increasing sampling yield without substantially affecting damage to the tissue. The regularity or periodicity of the pillar structure facilitates manufacture and reproducibility. The plurality of protrusions may be arranged in a substantially uniform grid pattern, or an offset pattern.

In one embodiment, the protrusions are formed on one or more tubular sheaths arranged on the distal portion of the elongated member. In this way, the protrusions may be formed on a separate component from the elongated member, thereby simplifying manufacture and enabling use of different materials for the sheaths and the elongated member.

In one embodiment, each protrusion comprises a sharp edge oriented in a tangential direction and/or longitudinal direction of the elongated member. The sharp edges increase the effectiveness of sampling through shearing or cutting. The sharp edge may form an overhang defining a collecting cavity adjacent each protrusion. Sampled cells or tissue may be retained in the collecting cavities during retraction of the biopsy device.

In one embodiment, at least the distal portion of the elongated member is made of a superelastic material. Superelasticity, especially shape-memory alloys such as nickel titanium, allows the elongated member to adopt the shape of the vasculature when the device is inserted therein.

In one embodiment, the protrusions are formed in a single monolithic structure. The structure may include the surface on which the protrusions are formed. The single monolithic structure provides the desired rigidity of the protrusions.

In one embodiment, the protrusions are formed by additive manufacturing. Additive manufacturing or 3D printing allows for high precision construction of the desired configuration of protrusions not possible with other manufacturing techniques.

In one embodiment, the protrusions are made of a polymer such as polyether, polyamide, polyimide or polytetrafluoroethylene, PTFE, or a metal.

In one embodiment, a distal tip of the tubular member and/or elongated member is sharp. The sharp distal tip enables the tubular member and/or elongated member to penetrate tissue to reach and sample desired target areas in the subject.

In a second aspect of the present disclosure, there is provided a method for sampling endothelial cells or tissue from a blood vessel or lymphatic vessel in a subject, the method comprising:
providing a tubular member, and an elongated member movably arranged within a lumen of the tubular member, wherein a distal portion of the elongated member comprises a tissue capture arrangement including a plurality of rigid protrusions;
introducing the tubular member into the vasculature of the subject to reach a target blood vessel or lymphatic vessel;
advancing the elongated member such that the distal portion of the elongated member exits the tubular member;
moving the elongated member such that the protrusions scrape the inner wall of the blood vessel or lymphatic vessel;
retracting the elongated member into the lumen of the tubular member; and retracting the tubular member from the vasculature.

In one embodiment, the step of advancing comprises advancing the elongated member until the distal portion of the elongated member becomes wedged inside the blood vessel or lymphatic vessel and the step of moving comprises rotating the elongated member. By wedging the distal portion of the elongated member inside the vessel, the protrusions are forced into contact with the vessel wall. Rotation of the elongated member in this wedged position ensures that the protrusions scrape the inner wall to sample endothelial cells with great yield whilst remaining minimally invasive. To this end, the outer diameter of the distal portion of the elongated member may be larger than the inner diameter of the vessel.

### BRIEF DESCRIPTION OF DRAWINGS

The disclosure is now described, by way of example, with reference to the accompanying drawings, in which:

FIG. 1 shows top, side and isometric views, respectively, of a biopsy device in the form of a micrograter 10.

FIG. 2 shows isometric views of alternative embodiments of a micrograter 10.

FIG. 3 shows isometric,side and cross-sectional views, respectively, of a distal portion of an elongated member having a plurality of micrograters 10 mounted thereon.

FIGS. 4 and 5 show isometric,side and cross-sectional views, respectively, of a biopsy device in the form of a spiral device.

FIG. 6 shows isometric,side and cross-sectional views, respectively, of a biopsy device comprising corrugations.

FIGS. 7a and 7b show isometric views of alternative embodiments of a biopsy device.

FIG. 8 shows cross-sectional views illustrating sampling cells in a blood vessel using a biopsy device comprising a micrograter 10.

FIG. 9 shows isometric,side and cross-sectional views, respectively, of a biopsy device comprising a plurality of protrusions.

FIG. 10 shows a side view of the biopsy device of FIG. 10 mounted on the distal portion of an elongated member movably arranged in a tubular member.

FIG. 11 shows an isometric view of the biopsy device of FIG. 10 mounted on the distal portion of an alternative elongated member movably arranged in an alternative tubular member.

FIG. 12 shows an X-ray guided pathway in the vasculature of a subject used by a biopsy device according to one embodiment of the present disclosure, an X-ray image of the biopsy device at a sampling location as well as different steps of operating the biopsy device.

### DESCRIPTION OF EMBODIMENTS

In the following, a detailed description of a device according to the present disclosure is presented. In the drawing figures, like reference numerals designate identical or corresponding elements throughout the several figures. It will be appreciated that these figures are for illustration only and are not in any way restricting the scope of the disclosure.

In the context of the present disclosure, it is understood that the terms “distal” and “distally” refer to a position or direction (furthest) away from the operator when using the device according to the present disclosure. Correspondingly, the terms “proximal” and “proximally” refer to a position or direction closest to or towards the operator when using the device according to the present disclosure.

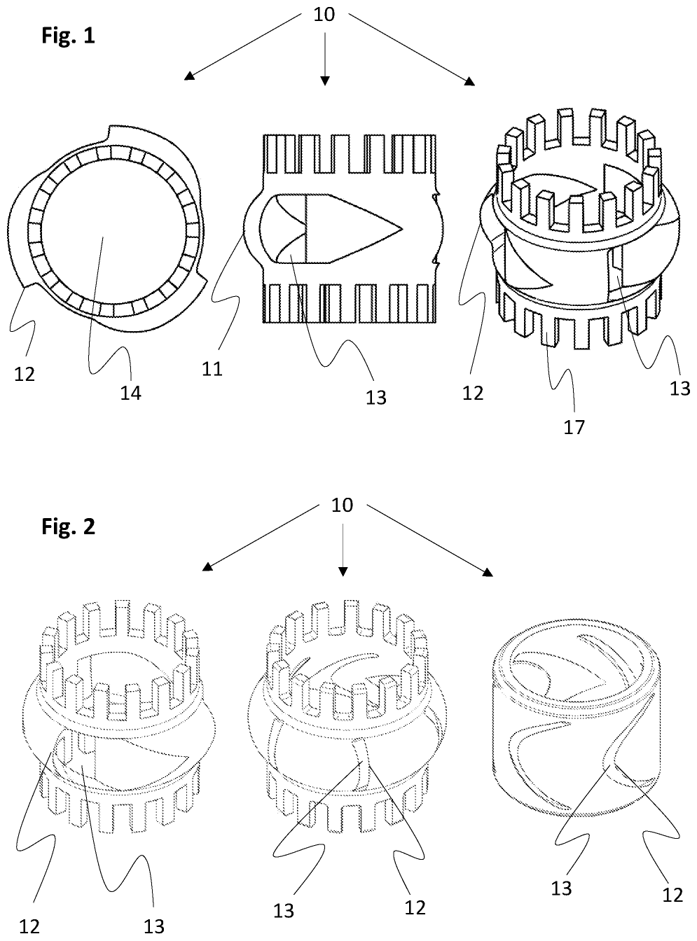

Referring to FIG. 1, there is shown one embodiment of a tissue capture arrangement in the form of a micrograter 10 in top, side and isometric views, respectively. The micrograter 10 is rotationally symmetric and has a tubular shape with a gradually increasing outer diameter, reaching the maximum at the mid-section 11, and decreasing afterwards. The micrograter 10 comprises rigid protrusions in the form of three raised sharp edges 12, aligned tangentially on the outer perimeter which form channels 13 leading into its lumen 14. The number of channels 13 are, for example, between 0 and 25 in number, preferably between 2 and 10, advantageously between 3 and 6. In one embodiment, the sharp edges 12 are oriented in a tangential direction. As such, the sharp edges 12 will scrape against the vessel wall only when rotated, not when advanced and retracted, thereby being minimally invasive. In other embodiments, the sharp edges may be oriented in a longitudinal or axial direction, or a combination of tangential and axial.

The micrograter 10 may be mounted on the outside of an elongated member 15 using glue, welding or other suitable fastening means. The elongated member 15 may be a wire or a tube and may comprise holes 16 or cavities, aligned with the channels 13. In one embodiment, the elongated member 15 is in the form of a 380 μm Nitinol tube which has laser cut holes 16 matching the channels 13 of the micrograter 10. Every grater edge 12 is thus connected to the inside of the Nitinol tube. The axial edges of the micrograters 10 contain a serrated surface with teeth 17 for providing extra surface area for the glue. The micrograters 10 are roughly 500 μm long, with an outer maximum diameter of 500 μm, and an inner diameter of 400 μm, creating a good fit with the Nitinol tube.

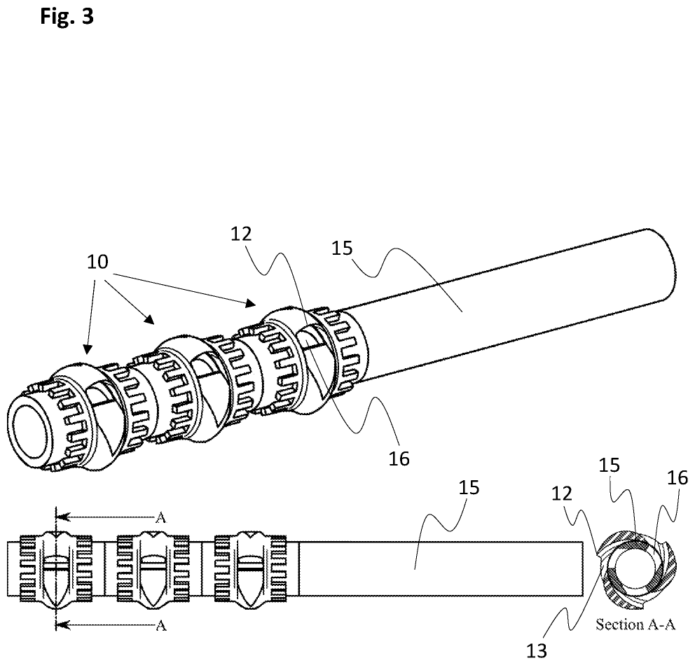

Referring now to FIG. 2, additional embodiments of a micrograter 10 are shown, having larger or smaller channels 13 and different shapes of the sharp edges 12, i.e. straight or arcuate, as well as with or without serrated axial end surfaces.

In one embodiment, shown in FIG. 3, three micrograters 10 are mounted on an elongated member 15 with an axial distance therebetween. The axial distance may be 0-1 mm, i.e. end-to-end or spaced apart. The distal end of the elongated member 15 may be covered by a cap (not shown), to prevent penetrating the vessel wall by mistake. The cap is configured to significantly increase penetration force and to prevent blood flow into the elongated member 15.

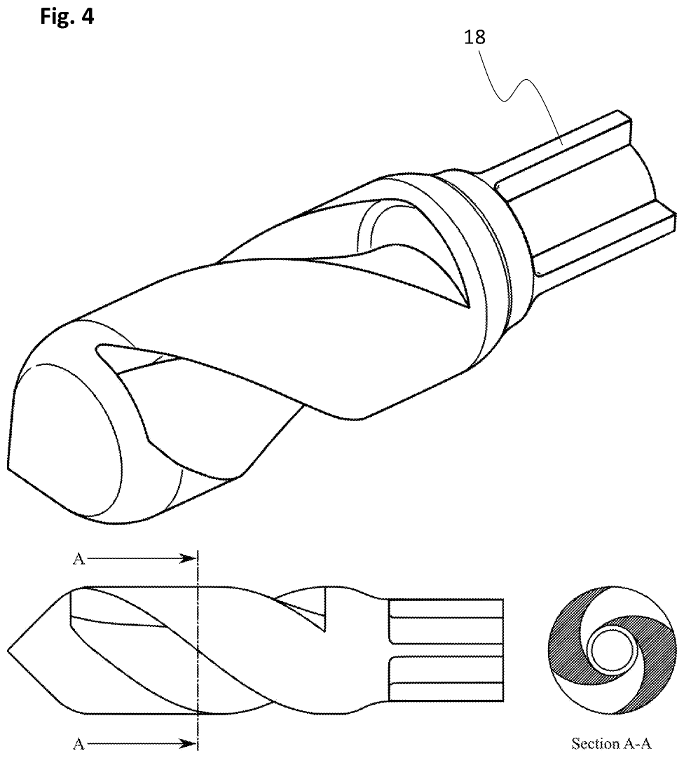

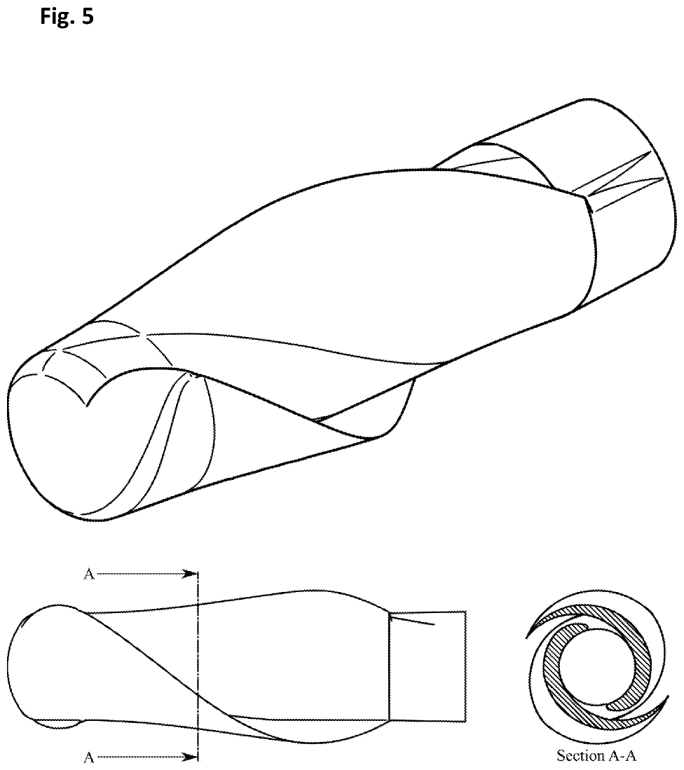

Referring now to FIGS. 4 and 5, there is shown another embodiment of a tissue capture arrangement comprising spiral flutes for capturing cells or tissue, and configured to be mounted on the distal end of the elongated member by means of a mounting interface 18, which may co-operate in an interlocking manner. The flutes define channels which may or may not be in fluid communication with the interior lumen of the biopsy device as shown in the cross-sectional view at the lower right of FIGS. 4 and 5. The distal end may be sharp as in FIG. 4 for penetrating tissue, or blunt as in FIG. 5.

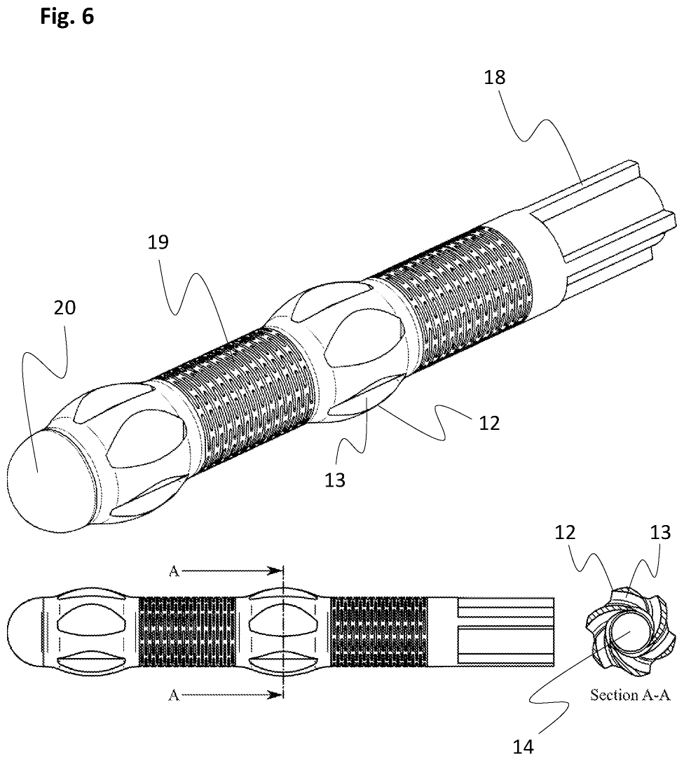

FIG. 6 shows another embodiment of a tissue capture arrangement comprising two sets of protrusions similar to the micrograter 10 shown in FIG. 1, in the form of sharp edges 12 and channels 13. The channels 13 may or may not be in fluid communication with the interior lumen 14. Adjacent the protrusions, there are areas 19 with a corrugated surface. The distal end of the biopsy device may be covered by a cap 20. The tissue capture arrangements of FIGS. 4-6 differ from the tissue capture arrangements of FIGS. 1-3 in that the former comprise a mounting interface at a proximal end configured to be attached to the distal end of an elongated member.

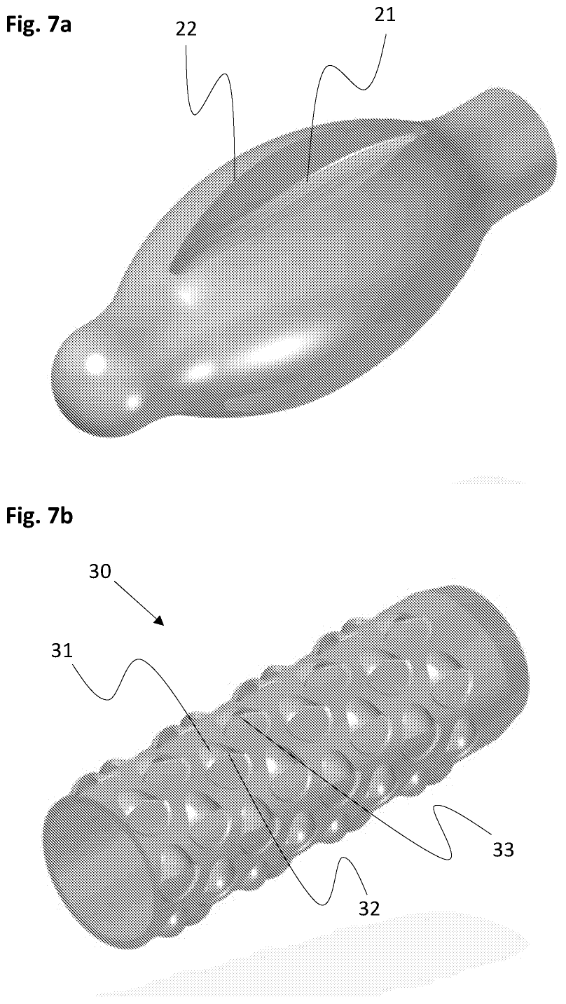

Referring now to FIGS. 7a and 7b, there is shown additional embodiments of a tissue capture arrangement. The tissue capture arrangement of FIG. 7a has an oval shape and comprises a gradually increasing and decreasing diameter and recesses 21 forming sharp edges 22. The number of recesses 21 are, for example, between 0 and 25 in number, preferably between 2 and 10, advantageously between 3 and 6.

The tissue capture arrangement of FIG. 7b is in the form of a tubular sheath 30 with a plurality of protrusions 31 formed on the outer surface. The protrusions 31 comprise sharp edges 32 oriented in a tangential and/or longitudinal direction and forming an overhang to define recesses or collecting cavities 33 adjacent each protrusion 31. The protrusions 31 are substantially regularly distributed about the circumference and along the length of the tubular sheath 30. The protrusions 31 are offset in relation to adjacent protrusions 31 in the axial direction.

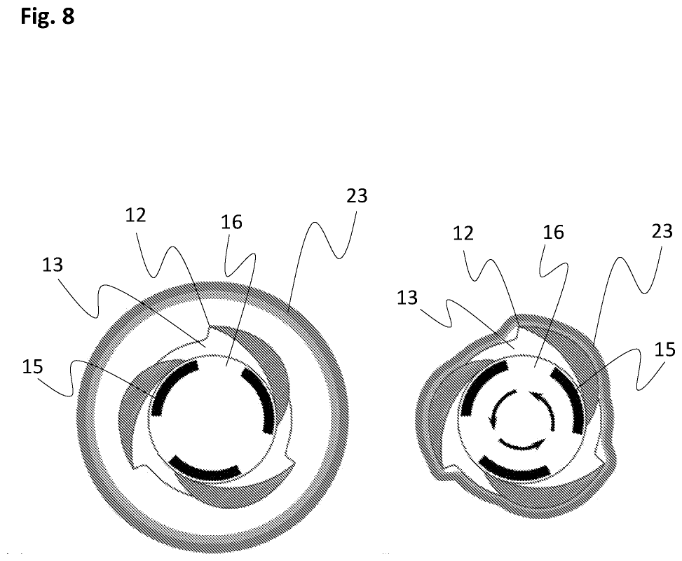

The biopsy device according to the present disclosure is designed to be navigated inside the vasculature, aided by X-ray guidance. Referring now to FIG. 8, there is shown in a cross-sectional view operation of the biopsy device in blood vessels of differing diameters. The biopsy device lies protected inside a tubular member (not shown), e.g. a guiding catheter, until a vessel of the correct diameter is reached, whereafter the elongated member 15 is advanced out of the lumen of the guiding catheter and wedged into the vessel. In a large vessel, as shown on the left side of FIG. 8, there is little or no contact between the tissue capture arrangement and the vessel wall 23. In a vessel of suitable diameter, ideally a bit smaller than the diameter of the tissue capture arrangement, as shown on the right side of FIG. 8, the sharp edges 12 of the tissue capture arrangement is forced into contact with the vessel wall 12 through wedging. The axial profile of the micrograters 10 outer diameter ensures a smooth interaction with the blood vessel wall when navigating to the target site. Once in a wedged position the elongated member 15 is manually rotated as shown by the arrows to facilitate the cell grating. When retracting, the elongated member 15 is pulled into the guiding catheter, protecting the sample, whereafter the entire biopsy device is withdrawn. The sampled tissue will be wedged into the sampling channels 13 and any blood that contaminates the sample can be flushed away after device withdrawal, without flushing away the tissue. Said tissue sample may have a volume of less than 50 μl, preferably less than 10 μl, advantageously less than 500 nl.

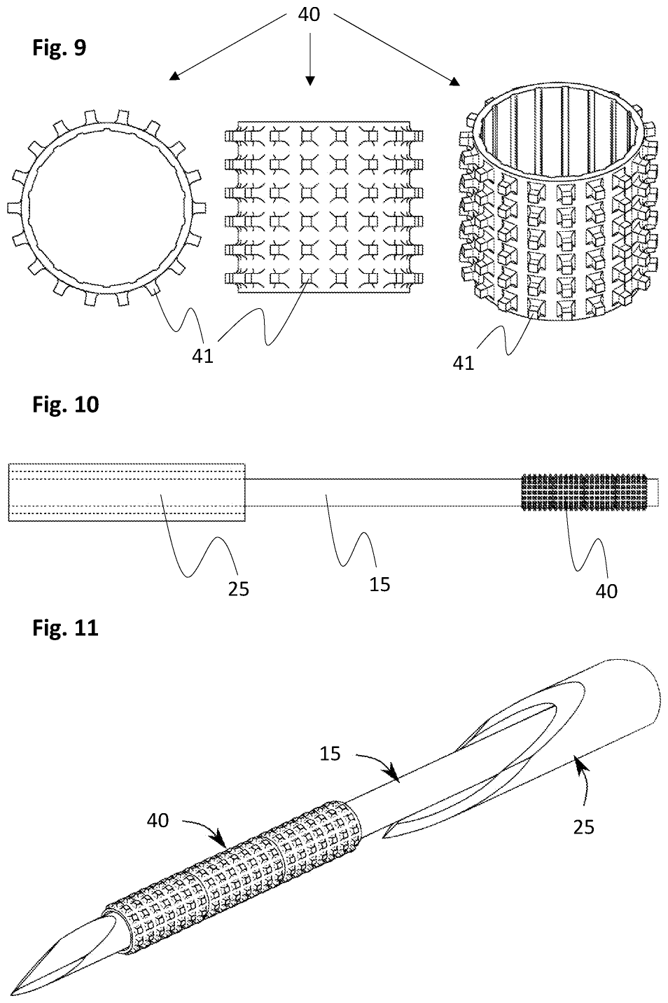

Referring now to FIG. 9, there is shown one embodiment of a tissue capture arrangement according to the present disclosure in top, side and isometric views, respectively. The tissue capture arrangement is in the form of a tubular sheath 40, similar to the one shown in FIG. 7b. On an outer surface of the tubular sheath 40, there is provided a plurality of rigid protrusions 41. As explained above, the protrusions 41 are rigid in the sense that they sufficiently stiff to resist movement in relation to the tubular sheath 40 and to each other. Moreover, the protrusions 41 are substantially incompressible such that they do not deflect or compress when contacted. In one embodiment, the protrusions 41 are formed in a single monolithic structure, i.e. the tubular sheath 40 and the protrusions 41 are made of the same material. In other embodiments, the protrusions 41 may be made of a different material than the tubular sheath 40. Suitable materials for the protrusions 41 include polymer, such as polyether, polyamide, polyimide or polytetrafluoroethylene, PTFE, or metal.

As may be seen in FIG. 9, the height of the protrusions 41 in the radial direction, perpendicular to the surface of the tubular sheath 40, is substantially smaller than the diameter of the tubular sheath 40, which corresponds to the diameter of the elongated member on which the tubular sheath 40 is arranged to be mounted. The ratio between the radial height of the protrusions 41 and the diameter of the tubular sheath 40 and/or elongated member 15 may be between 1:3 and 1:20, such as 1:4, 1:5, 1:6, 1:7, 1:8, 1:9, 1:10, 1:11, 1:12, 1:13, 1:14, 1:15, 1:16, 1:17, 1:18, 1:19. The radial height may be in the range 3-100 μm, preferably smaller than 50 μm, more preferably smaller than 20 μm, most preferably smaller than 10 μm. In one embodiment, the radial height of the protrusions 41 substantially corresponds to the dimension of the cells or tissue to be sampled.

In one embodiment, the plurality of protrusions 41 comprises a structure of pillars or blocks, as may be seen in FIG. 9. The pillars may be shaped like cubes, cylinders, pyramids or any other suitable shape. The pillars may be regularly distributed around the circumference and/or the length of the tubular sheath 40. In one embodiment, the pillars are arranged in a substantially uniform pattern exhibiting a repeated regularity or periodicity, such as a grid pattern shown in FIG. 9, or in an offset pattern like the protrusions 31 in the tissue capture arrangement shown in FIG. 7b. The periodicity facilitates the manufacturing process, for instance by additive manufacturing processes (3D printing).

Like the radial height, the distance between adjacent protrusions 41 may be adapted to the dimension of the cells or tissue to be sampled in order to increase sample yield. The distance may be in the range 3-100 μm, preferably smaller than 50 μm, more preferably smaller than 20 μm, most preferably smaller than 10 μm. In one embodiment, the ratio between the distance between adjacent protrusions 41 and the radial height is in the range 1:2 to 5:1.

In one embodiment, the width of the pillars corresponds to the radial height, i.e. they are substantially equal in dimension. As such, the width may be in the range 3-100 μm, preferably smaller than 50 μm, more preferably smaller than 25 μm, most preferably smaller than 15 μm. In one embodiment, the ratio between the width and the radial height of the protrusions 41 is in the range 1:2 to 2:1.

The effective diameter of the tubular sheath 40 may be smaller than 20 mm, such as 100 μm to 2 mm, more preferably 500 μm to 1 mm, most preferably smaller than 600 μm. The length of the tubular sheath 40 may be substantially equal to the diameter.

Referring now to FIG. 10, there is shown a biopsy device according to the present disclosure in a side view. The biopsy device comprises a tubular member 25 such as a catheter, needle or cannula and may be flexible for navigating through the vasculature, or more or less rigid for sampling closer to the skin. Inside the lumen of the tubular member 25, an elongated member 15 is movably arranged, i.e. the elongated member 15 may be advanced and retracted in the longitudinal direction in relation to the tubular member 25. The elongated member 15 may be wholly or partially made of a superelastic material, such as a nickel titanium alloy.

The distal portion of the elongated member 15 comprises a tissue capture arrangement including a plurality of rigid protrusions 41, here in the form of one or more tubular sheaths 40 as shown in FIG. 9. However, any of the tissue capture arrangements described above may be combined with the elongated member 15 to form the biopsy device. It is also foreseen that the plurality of protrusion 41 may be formed directly on the elongated member 15.

Referring now to FIG. 11, another embodiment of a biopsy device is illustrated in an isometric view. Here, the distal ends of both the tubular member 25 and the elongated member 15 comprise a sharp tip or leading edge for penetrating and/or cutting tissue. Said sharp leading edge preferably has an edge radius less than 50 μm, more preferably less than 5 μm.

This embodiment is similar to the biopsy device and method for tissue sampling in mammals discloses in WO/2021/137746 A1, the contents of which are incorporated herein by reference. More particularly, the endoluminal access device in the form of a flexible elongated hollow body terminating in a distal penetration portion arranged to penetrate a vascular tissue wall may be used in conjunction with the biopsy device of the present disclosure when sampling tissue in or via a blood vessel.

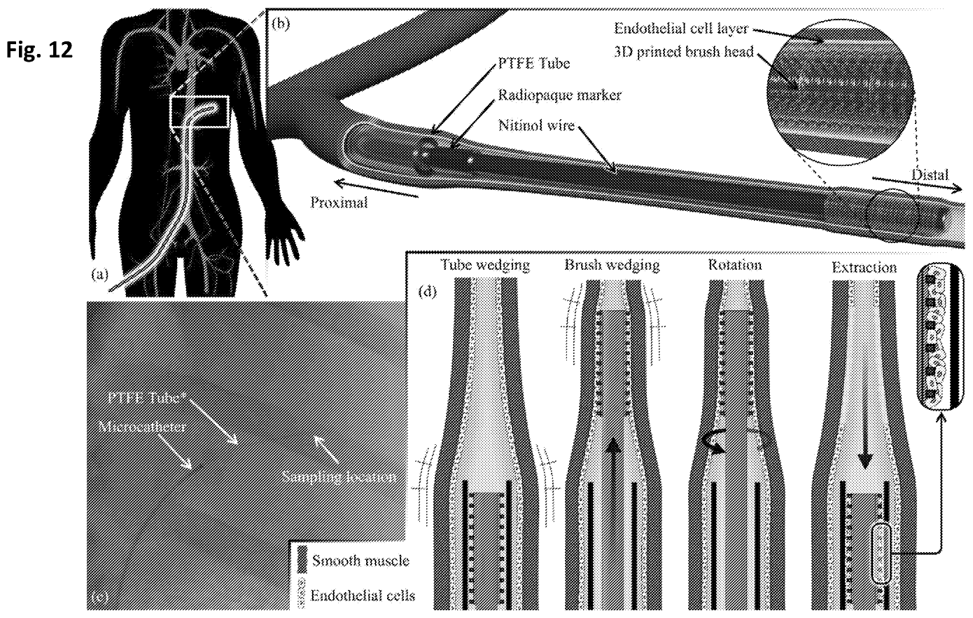

Referring now to FIG. 12, in drawing (a) there is shown a schematic view of an X-ray guided biopsy procedure in the vasculature of a subject to sample endothelial cells. A biopsy device in accordance with the present disclosure is inserted into the vasculature of the patient to reach a target blood vessel. At the target sampling location, the elongated member (Nitinol wire) is advanced through the lumen of the tubular member (PTFE tube) in a distal direction as shown in drawing (b). An X-ray image of the biopsy device is shown in drawing (c). A radiopaque member may be provided on the Nitinol wire to improve visibility during X-ray imaging.

In drawing (d) of FIG. 12, steps of a procedure for sampling endothelial cells are shown. The PTFE tube is advanced to the target sampling location until the tube becomes wedged. Then the Nitinol wire is advanced such that the tissue capture arrangement (3D printed brush head) on the distal portion exits the tube until the brush becomes wedged in the blood vessel. Next, the Nitinol wire is rotated in the wedged position which causes the brush to rotate and abrade cells from the vessel wall, which are then captured between the protrusions 41 of the brush. Then, the Nitinol wire is retracted into the lumen of the tube and the tube is retracted from the vasculature.

### EXAMPLE

### Device Design

The assembled catheter consists of a Nitinol tube, along which are place rotationally symmetric graters. Each grater has three sharp edges, aligned tangentially on the outer perimeter which form channels leading into its lumen. The micrograter has a gradually increasing outer diameter, reaching the maximum at the mid-section, and decreasing afterwards. The micrograter is glued to the outside of a 380 μm Nitinol tube which has laser cut holes matching the channels of the device. Every grater edge is thus connected to the inside of the Nitinol tube. The axial edges of the micrograters contain a serrated surface for providing extra surface area for the glue. The micrograters are roughly 500 μm long, with an outer maximum diameter of 500 μm, and an inner diameter of 400 μm, creating a good fit with the Nitinol tube.

A total of five micrograters are mounted on the Nitinol tube with 1 mm axial distance. The Nitinol tube is covered by a 3D-printed cap, to prevent penetrating the vessel wall by mistake. The system is designed to be navigated inside the vasculature, aided by x-ray guidance. The device lies protected inside a guiding catheter until a vessel of the correct diameter is reached, whereafter the device is protruded and wedged into the vessel. The axial profile of the micrograters outer diameter ensures a smooth interaction with the blood vessel wall when navigating to the target site. Once in a wedged position the device is manually rotated to facilitate the cell grating. When retracting, the device is pulled into the guiding catheter, protecting the sample, whereafter the entire system is withdrawn. The sampled tissue will be wedged into the sampling channels and any blood that contaminates the sample can be flushed away after device withdrawal, without flushing away the tissue.

### Fabrication and Assembly

Micrograters were printed with the Nanoscribe Photonic Professional GT2 in TP-S resin using a 25× objective, with a solid inner hatching, and standardized laser parameters. Nine micrograters were printed in 3×3 arrays, with a total print time of 5 hours and 19 minutes. The micrograters were developed in PGMEA and IPA for 20 and 5 minutes, respectively. The micrograters were aligned to the Nitinol tube using a microscope with an XY-stage for guidance. They are axially aligned to the laser cut openings and subsequently fixated to the Nitinol tube using cyanoacrylate glue.

In-vivo cell sampling was performed in a porcine model. The devices were navigated to the outermost vessels of the liver, into a position where there was no more blood flow. Successful cell retrieval was demonstrated by DAPI staining of the fixated catheters after sampling.

The devices acquired large amounts of tissue, indicating good likelihood that some of the tissue surrounding the endothelial cell layer has been sampled as well.

Preferred embodiments of a device for detaching cells or tissue from a cavity in a subject according to the present disclosure has been described. However, the person skilled in the art realizes that these can be varied within the scope of the appended claims without departing from the inventive idea.

All the described alternative embodiments above or parts of an embodiment can be freely combined without departing from the inventive idea as long as the combination is not contradictory.

1. A biopsy device for sampling cells or tissue in a subject, the biopsy device comprising:
a tubular member defining a lumen; and
an elongated member movably arranged within the lumen of the tubular member, wherein a distal portion of the elongated member comprises a tissue capture arrangement including a plurality of rigid protrusions.

2. The biopsy device according to claim 1, wherein a height of the protrusions in a radial direction is substantially smaller than a diameter of the elongated member.

3. The biopsy device according to claim 2, wherein a ratio between the radial height of the protrusions and the diameter of the elongated member is between 1:3 and 1:20.

4. The biopsy device according to claim 1, wherein the radial height of the protrusions is in the range 3-100 μm.

5. The biopsy device according to claim 1, wherein the plurality of protrusions comprises a structure of pillars regularly distributed around the circumference of the elongated member and/or along the length of the distal portion.

6. The biopsy device according to claim 5, wherein the plurality of protrusions is arranged in a substantially uniform grid pattern or an offset pattern.

7. The biopsy device according to claim 1, wherein a ratio between a distance between adjacent protrusions and a radial height of the protrusions is in the range 1:2 to 5:1.

8. The biopsy device according to claim 1, wherein a ratio between a width and a radial height of the protrusions is in the range 1:2 to 2:1.

9. The biopsy device according to claim 1, wherein the protrusions are formed on one or more tubular sheaths arranged on the distal portion of the elongated member.

10. The biopsy device according to claim 1, wherein each protrusion comprises a sharp edge oriented in a tangential direction and/or longitudinal direction of the elongated member.

11. The biopsy device according to claim 10, wherein the sharp edge forms an overhang defining a collecting cavity adjacent each protrusion.

12. The biopsy device according to claim 1, wherein at least the distal portion of the elongated member is made of a superelastic material.

13. The biopsy device according to claim 1, wherein the protrusions are formed in a single monolithic structure.

14. The biopsy device according to claim 1, wherein the protrusions are formed by additive manufacturing.

15. The biopsy device according to claim 1, wherein the protrusions are made of a polymer such as polyether, polyamide, polyimide or polytetrafluoroethylene (PTFE) or a metal.

16. The biopsy device according to claim 1, wherein a distal tip of the tubular member and/or elongated member is sharp.

17. A method for sampling endothelial cells or tissue from a blood vessel or lymphatic vessel in a subject, the method comprising:
providing a tubular member, and an elongated member movably arranged within a lumen of the tubular member, wherein a distal portion of the elongated member comprises a tissue capture arrangement including a plurality of rigid protrusions;
introducing the tubular member into the vasculature of the subject to reach a target blood vessel or lymphatic vessel;
advancing the elongated member such that the distal portion of the elongated member exits the tubular member;
moving the elongated member such that the protrusions scrape the inner wall of the blood vessel or lymphatic vessel;
retracting the elongated member into the lumen of the tubular member; and
retracting the tubular member from the vasculature.

18. The method according to claim 17, wherein the step of advancing comprises advancing the elongated member until the distal portion of the elongated member becomes wedged inside the blood vessel or lymphatic vessel and the step of moving comprises rotating the elongated member.
